(19) United States
(12) Patent Application Publication
(10) Pub. No.: US 2024/0075316 A1
(43) Pub. Date: Mar. 7, 2024

(54) RADIOTHERAPY APPARATUS WITH OPTIMISED DETECTOR

(71) Applicant: Elekta Limited, London (GB)

(72) Inventor: David Roberts, London (GB)

(21) Appl. No.: 18/260,080

(22) PCT Filed: Dec. 17, 2021
(86) PCT No.: PCT/GB2021/053348

§ 371 Date: Jun. 30, 2023

(30) Foreign Application Priority Data
Dec. 31, 2020 (GB) 2020871.6

(51) Int. Cl. A61N 5/10

(52) U.S. Cl. CPC A61N 5/1047; A61N 5/1075; A61N 2005/1076

(57) ABSTRACT

Disclosed herein is a radiotherapy apparatus comprising a radiation source configured to emit a beam of radiation having a central axis, a multi-leaf collimator, MLC, for shaping the beam of radiation emitted by the radiation source, wherein the MLC comprises a plurality of leaves, and a detection device for detecting radiation emitted by the radiation source. The detection device comprises a first detector arranged to detect a position of the central axis, wherein the first detector comprises a two dimensional array of pixels for generating a two dimensional map of radiation intensity, and at least one second detector arranged to detect a position of each leaf of the plurality of leaves.

The present disclosure relates to radiotherapy systems, and in particular to radiotherapy apparatus comprising a radiation source and a detection device for detecting radiation.

### BACKGROUND

Radiotherapy can be described as the use of ionising radiation, such as X-rays, to treat a human or animal body. Radiotherapy is commonly used to treat tumours within the body or skin of a patient or subject. In such treatments, ionising radiation is used to irradiate, and thus destroy or damage, cells which form part of the tumour.

Radiotherapy devices may have portal imaging capabilities. Such a radiotherapy device may comprise an integrated electronic portal imaging device (EPID). It is known to utilise integrated imaging devices, such as amorphous silicon image detectors, to perform tasks such as confirmation of the patient setup relative to the radiation beam and portal dosimetry (the measurement of the exit dose from a patient and subsequent reconstruction of delivered dose on the patient geometry). These detectors can also be used for checking the outputs of a radiotherapy device to provide quality assurance (QA) in relation to the device.

However, many of these integrated portal imaging devices are optimised for patient imaging using a megavoltage (MV) beam. They are also designed for high quantum efficiency to reduce radiation dose to the patient, which increases cost. The result is that a typical EPID detecting panel is large, has a very high sensitivity across its width and length, and is optimised to produce high resolution patient images. Such prior detecting panels are by necessity expensive, an issue which is compounded by the fact that, due to their presence in the path of the damaging high energy MV beam during treatment, they do not have a long operational lifetime and must be replaced often.

It would be advantageous to provide a radiotherapy apparatus with a radiation detection device which is optimised for performance and cost in relation to the task of performing quality assurance (QA) in relation to the apparatus.

The present invention seeks to address this, and other disadvantages encountered in the prior art, by providing an improved radiotherapy apparatus with a radiation detection device.

### SUMMARY

Aspects and features of the present invention are described in the accompanying claims.

According to an aspect, the present invention provides a radiotherapy apparatus comprising: a radiation source configured to emit a beam of radiation having a central axis; a multi-leaf collimator, MLC, for shaping the beam of radiation emitted by the radiation source, wherein the MLC comprises a plurality of leaves; a detection device for detecting radiation emitted by the radiation source, wherein the detection device comprises: a first detector arranged to detect a position of the central axis, wherein the first detector comprises a two dimensional array of pixels for generating a two dimensional map of radiation intensity; at least one second detector arranged to detect a position of each leaf of the plurality of leaves.

The first detector may have a higher resolution than the at least one second detector.

The radiotherapy apparatus may comprise a volume between the radiation source and the detection device in which the phantom may be placed to be irradiated.

The first detector may be configured to detect the position of the phantom when the phantom is positioned at or near an isocentre of the radiotherapy apparatus.

The first detector may be configured to detect the position of the phantom relative to the isocentre by imaging a projection of the phantom.

The radiotherapy apparatus may further comprise a controller configured to control the radiation source, the MLC and the detection device, in order to determine a position of the central axis of the beam of radiation with respect to the radiotherapy apparatus and to determine a position of each leaf of the MLC with respect to the radiotherapy apparatus.

The controller may be further configured to control the at least one second detector to a detect a profile of the beam of radiation.

The at least one second detector may be spaced apart from the first detector.

The first detector and the at least one second detector may be separated by at least one non-detecting region of the detection device.

The first detector may have a smaller pitch between pixels than the at least one second detector, thereby having a higher resolution.

The or each second detector may comprise a one dimensional array of sensors.

Each of a plurality of the sensors may be aligned with a respective leaf of the MLC in order to detect a position of the leaf.

The detection device may comprise at least two second detectors, thereby allowing the detection device to detect each leaf in at least two discrete positions.

A pitch between sensors may be the same as a pitch of the leaves of the MLC when projected to the detection device.

The at least one second detector may comprise two orthogonal second detectors configured to detect the profile of the beam of radiation in two dimensions.

The MLC and the detection device may be disposed in a fixed position relative to one another.

The MLC and the detection device may be provided at opposing sides of a rotatable gantry.

The first detector may be configured to determine the location of the central axis of the field of radiation at each of a plurality of gantry rotation angles, in order to allow determination of an isocentre position for the radiotherapy apparatus.

The detection device may be configured to provide dosimetry data for a dose of radiation delivered to a patient.

According to another aspect, the present invention provides a method of testing an operation of the radiotherapy apparatus of any preceding claim, the method comprising: controlling the radiation source to irradiate a phantom with the beam of radiation; and controlling the first detector to detect the position of the phantom.

The method may further comprise detecting a position of a leaf of the plurality of leaves of the MLC using the second detector.

The position of the leaf may be detected by controlling the MLC to move the leaf in the beam of radiation while detecting a projection of the leaf using the at least one second detector.

According to another aspect, the present invention provides a computer-readable medium comprising computer-executable instructions which, when executed by a processor, cause the processor to perform any of the methods disclosed herein.

### FIGURES

Specific embodiments are described below by way of example only and with reference to the accompanying drawings in which:

FIG. 1 illustrates a radiotherapy apparatus;

FIG. 2 depicts an example of a beam shaping apparatus;

FIG. 3 illustrates an embodiment of a detection device;

FIG. 4 illustrates another embodiment of a detection device;

FIG. 5 illustrates another embodiment of a detection device;

FIG. 6 illustrates another embodiment of a detection device;

FIG. 7 is a flow chart setting out a method for measuring an isocentre for the radiotherapy apparatus; and

FIG. 8 depicts a block diagram of a computing device.

### DETAILED DESCRIPTION

In overview, and without limitation, the present application relates to providing a radiotherapy apparatus which may comprise a detection device with multiple detectors, in which a primary detector may be placed centrally to enable detection of the beam central axis (or beam isocentre) and one or more secondary detectors placed such that a determination as to the location of the leaves of a multi-leaf collimator may be provided.

Rather than provide a large panel with high sensitivity and high resolution across its entire surface area, the present application may improve cost efficiency based on the configuration of the primary detector and secondary detector(s). In some implementations, the primary detector may include a high resolution imaging panel placed in the centre of the detection device, while the secondary detector may comprise one or more “strips” or rows of sensors, for example a one dimensional array of photodiodes, where each sensor may be aligned for measurement of a respective MLC leaf.

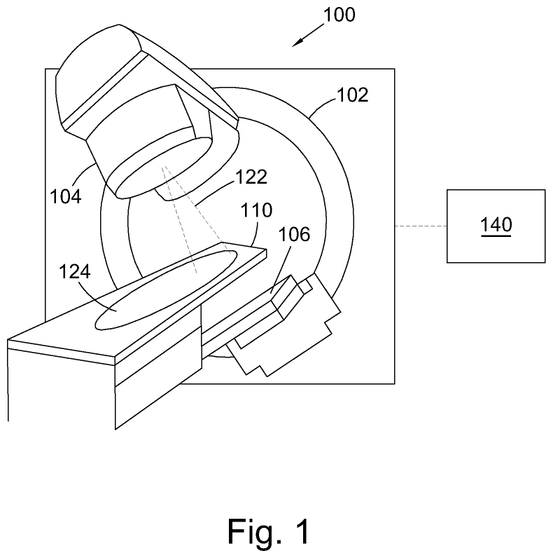

FIG. 1 is a view of an exemplary radiotherapy apparatus 100. Radiotherapy apparatus 100 is, for example, a linear accelerator (LINAC).

The radiotherapy apparatus 100 includes a gantry 102, which may support a radiation head 104 and a detection device 106. The radiation head 104 and the detection device 106 are mounted opposite each other on the gantry 102, with a rotational axis of gantry 102 positioned between them. The radiation head 104 is configured to generate a radiation beam 122 according to a treatment plan to deliver doses of radiation to a patient (or subject) 124 supported by a couch 110 (also referred to as a patient support surface or subject support surface). The gantry 102 is configured to rotate the radiation head 104 and detection device 106 about the couch 110, to provide patient 124 with a plurality of varying dosages of radiation according to the treatment plan.

The radiation head 104 may include a radiation source and a beam shaping apparatus for shaping the radiation beam 122. The radiation head 104 provides a beam of therapeutic radiation which may, for example, be in the megavoltage (MV) range. The beam shaping apparatus, described below in relation to the other Figures, may include a multi-leaf collimator (MLC).

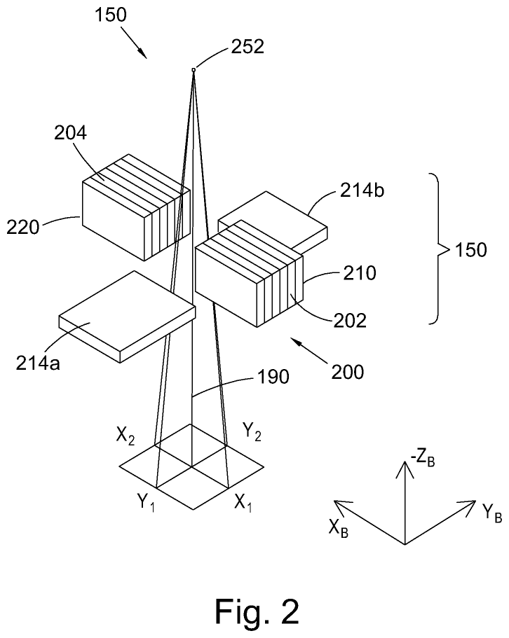

The radiation beam 122 has a beam central axis (also referred to simply as the beam axis) and a beam profile, which are described in more detail with reference to FIG. 2.

Positioned generally along a central axis of the gantry 102 is the couch 110 upon which a patient lies for radiotherapy. The couch 110 is configured to move with at least one degree of freedom, in order to allow positioning of a patient or QA device such as a phantom. For example, the couch can be moved in order to position the phantom at an isocentre of the radiotherapy apparatus, such that the radiation beam 122 is directed towards the phantom. The movement of the couch 110 is effected and controlled by a subject support surface actuator, which may be described as an actuation mechanism, which is controlled by the controller 140.

The couch 110 can be configured to support a phantom or other QA device at a location between the radiation head 104 and the detection device 106. For example, the location may be that of the assumed isocentre of the radiotherapy apparatus. The therapeutic radiation beam 122 is directed towards the detection device 106 such that the beam intersects the phantom during beam delivery. Radiation from the beam 122 which is incident upon the detection device 106 after the beam has intersected the phantom is detected by the detection device 106. The detected radiation varies in intensity due to the presence of the phantom in the path of the beam, and thus can be used to image the phantom in order to determine its projected location. The first detector may be configured to detect the position of the phantom relative to the isocentre by imaging a projection of the phantom.

The radiotherapy apparatus 100 includes a controller 140 which is programmed to control the radiation head 104, detection device 106, couch 110 and the gantry 102. Controller 140 may perform functions or operations such as treatment planning, treatment execution, image acquisition, image processing, motion tracking, motion management, and/or other tasks involved in a radiotherapy process.

The controller 140 is programmed to control features of the apparatus 100 according to a radiotherapy treatment plan for irradiating a target tissue of a patient. The treatment plan may include information about a particular dose to be applied to a target tissue, as well as other parameters such as beam angles, dose-histogram-volume information, the number of radiation beams to be used during therapy, the dose per beam, and the like. The controller 140 is programmed to control various components of apparatus 100—such as gantry 102, radiation head 104, detection device 106 and couch 110—according to the predetermined treatment plan.

The radiotherapy apparatus 100 has an isocentre. In an ideal system, the isocentre can be thought of as the point in space created by the intersection of the gantry rotation axis and the plane which is contains the trajectory of the radiation emitted by the source. This point serves as the geometric origin for the modelling of the radiation delivery in any treatment planning system and any patient imaging system. It is important to position a patient within the radiotherapy apparatus with knowledge of the position of the isocentre, and its position with respect to the target region. However, due to various effects (e.g. mechanical shifting or flexing as a function of gantry angle), the position of the radiation beam as defined by the beam shaping apparatus may also vary slightly with respect to this ideal isocentre as the apparatus is rotated.

The controller 140 can be configured to control QA procedures for the radiotherapy apparatus, by controlling the radiation head 104 and the detection device 106. This may include calibrating the positions of leaves of the MLC by controlling the movement of leaves and receiving measurements from the detection device 106.

The detection device 106 and controller 140 are adapted to measure the beam 122 of therapeutic radiation emitted by the radiation head 104. Detectors of the detection device 106 are specifically adapted to provide radiation intensity data in order to allow calibration, QA and other procedures to be performed. For example, the detection device 106 and controller 140 may be configured to perform MLC leaf calibration, measure the apparatus isocentre, and obtain dosimetry data for a dose of radiation delivered to a patient.

The detection device 106 advantageously includes an arrangement of detectors which may allow multiple QA procedures to be carried out, while reducing complexity and cost of the detection device itself. Additionally, the detection device 106 is integrated into the radiotherapy apparatus 100—being supported by the gantry 102 and connected to the controller 140. The detection device 106 can make measurements of the beam for QA purposes, where a patient is not generally present, and can also make measurements of the beam during a radiotherapy treatment, where a patient is present on the couch 110. The controller 140 can receive these measurements from the detection device 106 for use in QA procedures and, for example, dosimetry procedures.

The detection device 106 improves the layout of detectors in order to reduce cost. Additionally, the controller 140 can be configured to control the beam shaping apparatus and the detection device 106 in order to perform QA and calibration.

The detection device may include detectors with varying resolutions, which have been configured for QA/calibration/device setup purposes. For QA where a patient is not present, dose is not an issue and therefore the detector can be less efficient.

The detection device 106 and the radiation head 104 may be provided in a fixed spatial configuration relative to one another (e.g. by being mounted to opposing sides of the rotatable gantry 102). In such cases, it may thus not be necessary to manually position the detection device 106 in order to perform QA procedures, or the amount of manual positioning of the detection device 106 may be reduced.

The detection device 106 includes a first detector arranged to detect a position of the radiation beam axis with respect to the detection device. Detecting the central axis of the field of radiation is used to calculate the isocentre of the radiotherapy apparatus 100. Specifically, the first detector is configured to determine the location of the central axis of the field of radiation at each of a plurality of gantry rotation angles, thereby to allow determination of an isocentre position for the radiotherapy apparatus. The first detector can have a relatively high resolution in order to image the radiation beam and, for example, a ball bearing phantom. Since the central axis of the radiation beam 122 is generally incident on the detection device 106 in a central region of the detection device 106, the first detector does not need to extend over the whole area of the detection device 106. In some implementations, the first detector is provided in a central region of the detection device 106.

The detection device 106 may also include a second (also referred to as secondary) detector arranged to detect a position of each leaf of the MLC. The second detector may be provided as one or more elongate detectors comprising a series of sensors, where the sensors are aligned with respective leaves of the MLC. The second detector may have a lower resolution than the first detector. In some implementations, the second detector is provided in a peripheral region of the detection device 106.

The first (also referred to as primary) detector may have a two dimensional array of pixels for generating a two dimensional map of radiation intensity. The second (also referred to as secondary) detector may comprise one or more one dimensional arrays of sensors (e.g. diodes), each of which arranged to provide a measure of radiation intensity. Each sensor can be considered a pixel.

In this application, the resolution of detectors refers to the amount of radiation intensity information per unit area. This may include total pixels, pixels per unit area, and/or bits per pixel. Where detectors are described as one dimensional (e.g. the secondary detectors), this may correspond to a linear array of pixels. For the secondary detectors, each sensor (e.g. each diode) may correspond to a single pixel.

Advantageously, the detection device may be more resistant to radiation as it includes fewer active components compared to existing integrated panel detectors. Where the primary and/or secondary detectors include ionisation chambers, these can be less affected by the total dose received. Also, the cost of the detection device can be significantly reduced, e.g. the cost may be so low that it is economic to allow replacement on a periodic basis. The primary and secondary detectors may be individually replaceable.

FIG. 2 depicts an example of a beam shaping apparatus 150. FIG. 2 schematically depicts the position of a beam source 252 within the radiation source, from which radiation is produced, and schematically shows the beam passing through the beam shaping apparatus 150.

The beam shaping apparatus 150 includes a multi-leaf collimator, MLC, 200 and a diaphragm apparatus 214.

MLC 200 may include a plurality of elongate leaves 202, 204 oriented orthogonal to the axis 190 of beam 122. MLC 200 includes two banks 210, 220 of leaves, forming two opposing arrays. Each leaf can be individually extended into and out of the path of radiation beam 122 in order to shape the cross-section of the beam by blocking portions thereof. The leaves are movable in the longitudinal, or y, direction to provide shaping of the beam.

During radiotherapy treatment, the leaves of MLC 200 may be controlled to take different positions to selectively block some or all of radiation beam 122, thereby altering the shape of the beam that reaches the patient. In other words, the MLC presents an edge to the radiation beam which can be varied so as to provide a particular beam shape.

The radiation beam 122 has a beam central axis 190 (also referred to simply as the beam axis) and a beam profile. The radiation beam axis 190 is the axis at the centre of the beam at its maximum extent, when the beam shaping apparatus defines its maximum aperture. Therefore, the beam central axis may be defined by the geometry of the radiotherapy apparatus e.g. the edge of the central MLC leaves, or the geometric centre of the beam (e.g. the centre of the “window” defined by the MLC when all the leaves are retracted fully). The beam profile is the intensity distribution of the radiation on a plane perpendicular to the radiation beam axis 190. The beam profile can be measured using the detection device 106 as described below.

In some embodiments, beam limiting apparatus 150 may include a bank of motors, with each motor configured to move a corresponding one of the leaves.

Movement of each leaf by the motors is controlled by controller 140. For example, controller 140 controls leaf movement via the motors to shape radiation beam 122 for irradiating a target tissue, such as according to a treatment plan. Controller 140 moves the leaves, including advancing and retracting the leaves, by actuation of the leaf motors.

The beam limiting apparatus 150 also comprises a diaphragm apparatus. The diaphragm apparatus is configured to shape the beam of radiation, in a manner similar to the MLC 200. The diaphragm apparatus comprises one or more diaphragm blocks 214 configured to be extended into, and withdrawn from, the radiation field. In an example, the diaphragm apparatus comprises two diaphragm blocks 214a, 214b which face each other across the radiation field.

The diaphragm blocks 214a, 214b may be configured to move in a movement axis which is generally or substantially perpendicular to the beam axis 190, and also generally or substantially perpendicular to the movement axis of the MLC leaves. The diaphragm blocks 214a, 214b are made from a radiopaque material such as tungsten.

The beam limiting apparatus 150 further comprises diaphragm actuation means (not shown). In some implementations, the diaphragm actuation means includes a diaphragm motor, which is configured to effect movement of the diaphragm blocks 214a, 214b.

With reference to FIG. 2, it will be appreciated that actuation means of the MLC (e.g. the bank of motors) is configured to move the MLC leaves in the directions indicated as X1 and X2, and along a movement axis depicted in the figure as the ‘X’ direction. The diaphragm actuation means is configured to move the diaphragms in directions Y1 and Y2, and along a movement axis depicted in the figure as the ‘Y’ direction. The beam of radiation travels in the Z direction. While the diaphragm 214 depicted in FIG. 2 are positioned ‘underneath’ the MLC (i.e. farther away from the beam source 252), in alternative implementations the diaphragm may be positioned ‘above’ the MLC (i.e. closer to the beam source 252 than the MLC).

Considering the MLC, a first array 210 extends into the beam field in the X direction from one side of the field, and the second array 220 extends into the beam field in the X direction from the opposing side of the field. The leaves can each be moved independently to define a chosen shape between the tips of the opposing leaf banks 210, 220. Each leaf is thin in its transverse (Y) direction to provide good resolution, is deep in the z direction to provide adequate absorption, and long in its longitudinal (X) direction to allow it to extend across the field to a desired position.

Considering the diaphragm, movable blocks 214a and 214b adjust the width of the aperture. Specifically, the diaphragm blocks define the aperture in the Y direction. The leaves of the MLC can be fully extended such that directly opposing leaves meet. Solely using the MLC to define the beam width would constrain the width of the aperture is to integer numbers of the width of the MLC leaves. The diaphragm blocks 214a, 214b can be moved in the Y direction as desired, and therefore provide an unconstrained dimension of the beam width. Further, the tips of the leaves of the MLC can be curved and there may be some degree of leakage between the tips of directly opposing MLC leaves from opposing banks 210, 220 when fully extended to close off parts of the field. The diaphragm blocks 214a, 214b absorb radiation outside the desired width of the aperture to reduce leakage of the beam in locations outside the aperture.

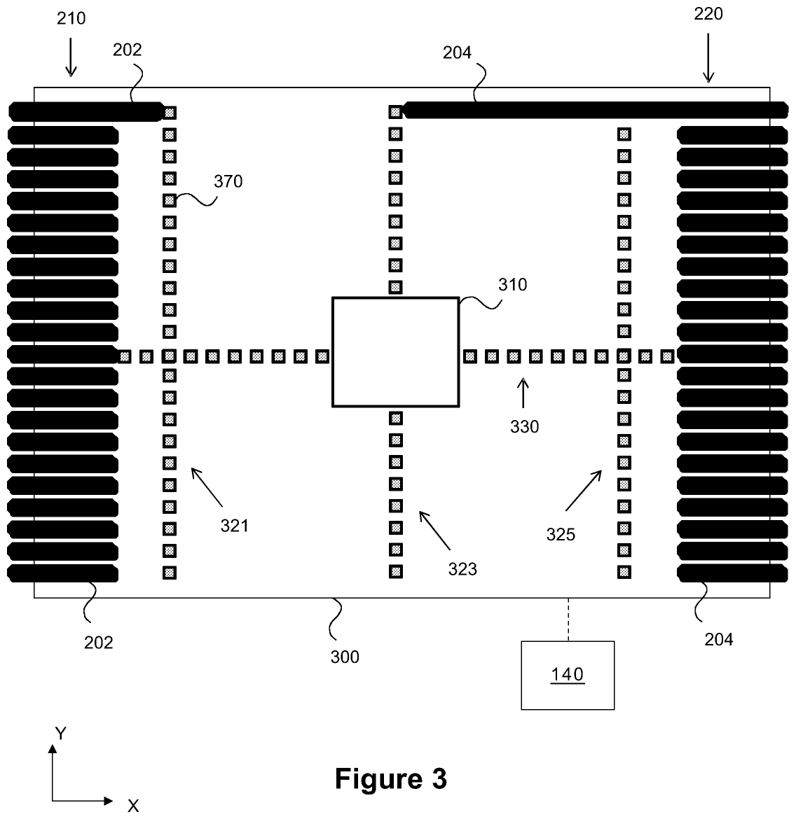

FIG. 3 illustrates an embodiment of a detection device 300, along with leaf banks 210, 220 of the MLC 200. FIG. 3 is a schematic depiction of how the MLC leaves are aligned when projected onto the detection device 300. The view shown in FIG. 3 can be thought of as showing the “shadows” of the MLC leaves on the detection device 300.

FIG. 3 shows the detection device 300 in the xy plane, where the beam axis (e.g. the z axis) goes into the page. The detection device comprises a primary detector 310 which is located in a central position on the detection device 300, such that the first detector 310 can detect the beam axis 190. Preferably, the first detector 310 is aligned with the beam central axis 190.

The size of the aperture (also referred to as detectable area) of the primary detector projected to isocentre is smaller than the maximum beam aperture (e.g. maximum aperture of the MLC) projected to isocentre. In fact, preferably the aperture of the primary detector projected to isocentre is also smaller than typical aperture sizes (projected to isocentre) of the beam shaping device used for radiotherapy.

The primary detector may comprise a photodiode array, for example having a photodiode pixel pitch of 3 mm, with 256 photodiodes (16×16) in total. The pixel size may be, for example, 2.5 mm×2.5 mm.

The detection device 300 can be used to detect the beam axis 190 at a number of different gantry rotation angles, which allows the isocentre to be determined by the controller 140. In operation, the primary detector 310 may be configured to detect the central axis of the radiation beam by imaging a ball bearing phantom. This can be done at different gantry angles and/or different radiation energies. The primary detector 310 provides a two dimensional image, which can image the position of the ball bearing phantom relative to the beam axis.

The primary detector is configured to provide a map (e.g. an image) of radiation intensity in two dimensions, whereas each of the one or more secondary detectors is configured to provide a map (e.g. an image) of radiation intensity in one dimension.

The primary detector may provide a relatively high resolution for imaging (e.g. a higher resolution than each of the secondary detectors).

The detection device 300 may also comprise four secondary detectors, including three secondary detectors 321, 323, 325 extending in the y direction, referred to as inline detectors, and one secondary detector 330 extending in the x direction, referred to as a crossline detector.

Each of the three inline detectors 321, 323 and 325 comprises a linear array of sensors 370. Each sensor is aligned with a respective pair of opposing MLC leaves 202, 204, such that the sensor can be used to determine a position of one or both on the MLC leaves 202, 204. One of the inline detectors 323 is aligned with the primary detector 310 (e.g. aligned with the centre of the primary detector in the x direction). The other two inline detectors 321, 323 are disposed on opposing sides of the detection device 300. Therefore, the inline detectors 321, 323, 325 allow every MLC leaf 202, 204 to be measured in three discrete positions.

The crossline detector 330 is aligned with the primary detector 310 (e.g. aligned with the centre of the primary detector in the y direction). The crossline detector 330 is configured to provide information on radiation intensity across a width of the beam field, thereby measuring a beam profile (e.g. beam profile in the x direction). At least one of the inline detectors 321, 323, 325 is also configured to provide information on radiation intensity across a width of the beam field, thereby measuring a beam profile (e.g. beam profile in the y direction). Providing two orthogonal secondary detectors in this way advantageously allows detection the profile of the beam of radiation in two dimensions.

Each sensor may comprise a diode for detecting radiation. In some implementations, some or all of the sensors comprise ion chambers or similar, instead of or in addition to the diodes.

The inline detectors are ideally suited to an MLC which is fixed in its orientation, because the leaves will always be aligned with the inline detectors.

The controller 140 is connected to the detection device 300, in order to receive signals from the detection device 300. The detection device 300 measures the intensity of radiation, and outputs corresponding signals to the controller 140 indicating the intensity of radiation at defined spatial positions on the detection device (e.g. radiation signal vs. position in the xy plane).

The controller is configured to move each leaf of the MLC and receive signals from the detection device in order to calibrate the MLC leaves. The position of a leaf is detected by controlling the MLC to move the leaf in the beam of radiation while detecting a projection of the leaf using one or more of the secondary detectors 321, 323, 325.

For example, the controller 140 may move a leaf in the x direction to align the leaf with a respective sensor on the detection device. In some embodiments, the controller determines that an MLC leaf is aligned with the respective sensor when the output signal from the sensor is, e.g., 50% of its maximum output (or another threshold). This indicates that the MLC leaf shadow extends half way across the sensor 370. Also, in some embodiments, the controller is configured to move an MLC leaf continuously across its movable range, and receive the output from the respective three sensors in the inline detectors 321, 323, 325 as the MLC leaf is moved. The 50% point between maximum and minimum radiation detected by each of the sensors 370 corresponds to alignment of the leaf with each of the respective inline detectors 321, 323, 325.

It can be seen in FIG. 3 that the central five pairs of MLC leaves 202, 204 are aligned with the primary detector 310 (this can vary in different implementations). The central inline detector 323 extends either side of the primary detector 310. For calibration of these central leaves, the peripheral inline detectors 321 and 325 are used in the same way as described above, however the primary detector 310 is used instead of the central inline detector 323. The primary detector 310 images the central leaves as they move, and the controller 140 uses these images to determine when the MLC leaves are aligned centrally with the primary detector 310.

The crossline detector 330 in combination with the central inline detector 323 (and optionally the primary detector 310 and/or peripheral inline detectors 321 and 325) are used to measure beam shape and symmetry. The detectors determine a profile of the beam in the x and y directions.

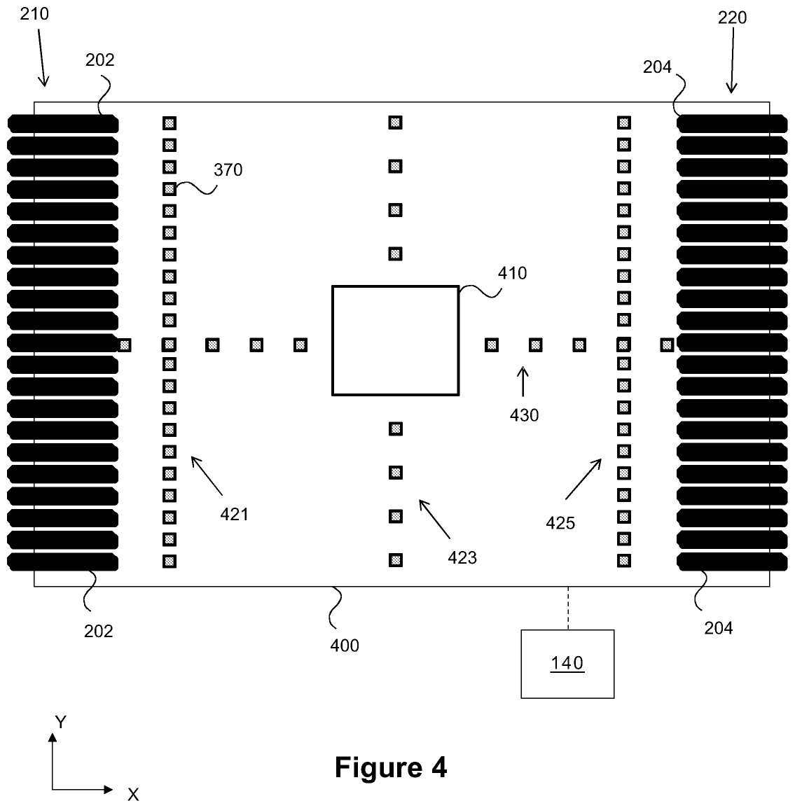

FIG. 4 illustrates another embodiment of a detection device 400, which is similar to the detection device 300 shown in FIG. 3. The detection device 400 includes a primary detector 410 which is similar to the primary detector 310 in FIG. 3 and therefore will not be described further. The detection device 400 includes three secondary inline detectors 421, 423, 425 and a secondary crossline detector 430. Two of the inline detectors 421 and 425 may be located remote from the primary detector 410 (e.g. they are peripheral to the primary detector 410). These two inline detectors 421, 425 are configured to detect the MLC leaves 202, 204 in two discrete positions. Their configuration is therefore similar to the inline detectors 321, 325 of the detection device 300 shown in FIG. 3.

In contrast to the detection device 300 shown in FIG. 3, the central inline detector 423 is not specifically configured for detecting the MLC leaves. The central inline detector 423 and the crossline detector 430, both of which intersect the primary detector 410, are configured to measure a beam profile. They have a lower resolution than the other secondary detectors 421, 425, and the primary detector 410. These detectors have a lower resolution (e.g. a lower density of sensors and/or greater pitch of sensors) because an acceptable beam profile can be obtained with a lower resolution than the resolution used for the inline detectors 421, 425 to detect the MLC leaves.

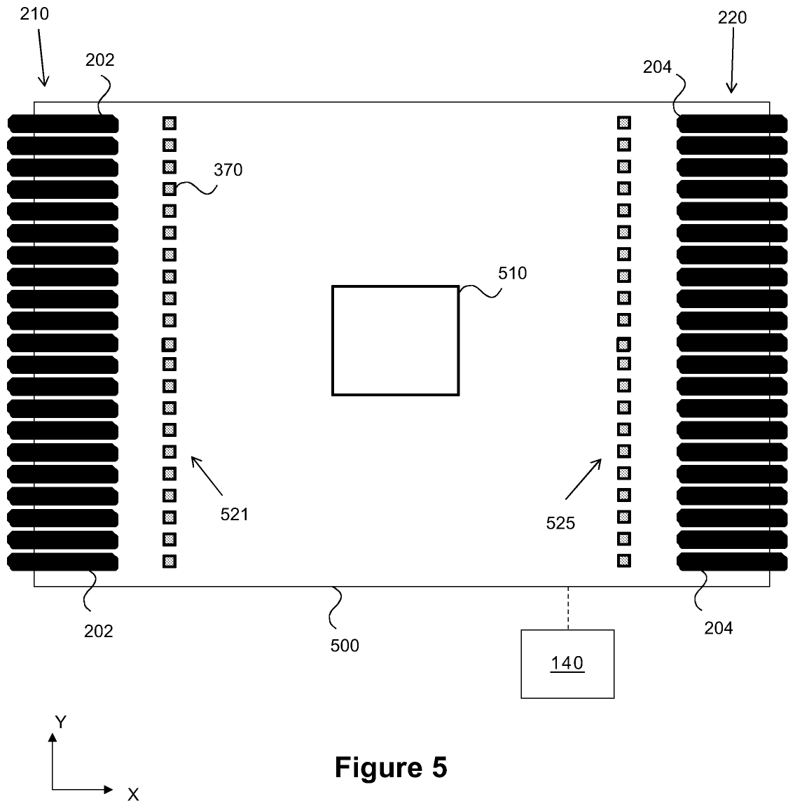

FIG. 5 illustrates another embodiment of a detection device 500. The detection device 500 includes a primary detector 510 which is similar to that of the primary detector 310 in FIG. 3 and therefore will not be described further. The detection device 500 also includes two secondary detectors 521, 525 which are located remote from the primary detector 510 (e.g. they are peripheral to the primary detector 510). These two inline detectors 521, 525 are configured to detect the MLC leaves 202, 204 in two discrete positions. Their configuration is therefore similar to the inline detectors of the detection device 400 shown in FIG. 4.

It can be seen from FIG. 5 that the primary detector 510 and the two secondary detectors 521, 525 are spaced apart from one another. The regions of the detection device 500 which separate the primary detector 510 and the two secondary detectors 521, 525 are non-detecting regions which are not configured to detect radiation and do not comprise any detecting elements. This advantageously means that the detection device 500 may be cheaper and may improve the detection of the beam axis and MLC leaf positions, since not all regions of the detection device 500 are detecting regions.

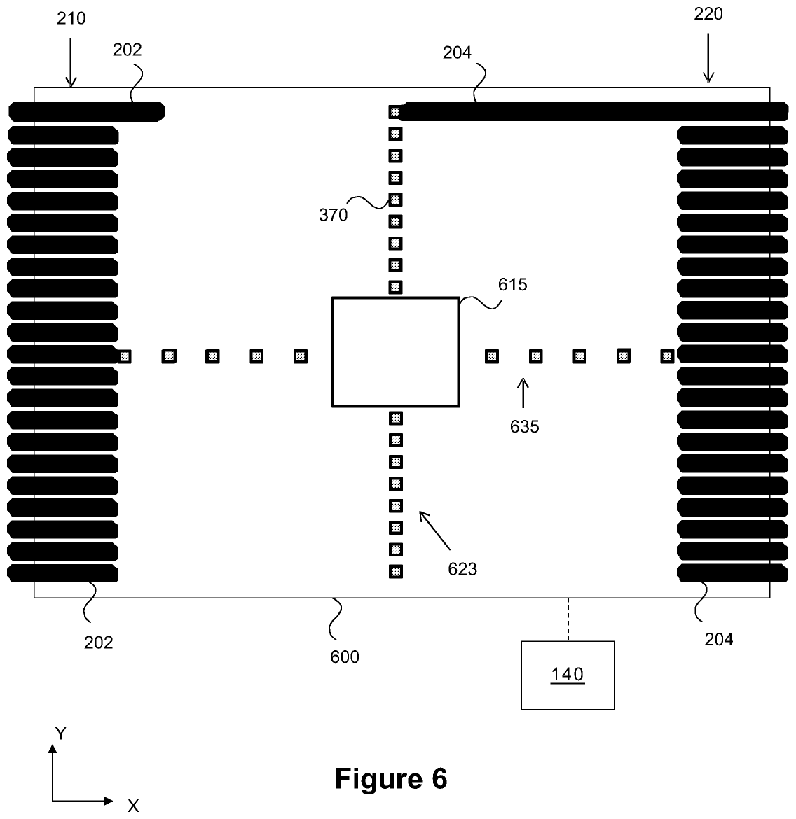

FIG. 6 illustrates yet another embodiment of a detection device 600. The detection device 600 includes a primary detector 615 which is similar to that of the primary detector 310 in FIG. 3 and therefore will not be described further.

The detection device 600 includes secondary detectors 623 and 635. Secondary detector 623 is an inline detector which is configured to detect each of the MLC leaves 202, 204 in a single position. Preferably, this position is halfway between the leaf banks 210, 220. The inline detector 623 is also configured to measure a beam profile of the radiation beam in the y direction.

Secondary detector 635 is a crossline detector configured to measure a beam profile of the radiation beam in the x direction. The secondary crossline detector 635 has a lower resolution than the inline detector 623.

The detection device can advantageously be used to provide dosimetry data in addition to detecting MLC leaf positions, beam axis etc.

The methods described herein may also be used as part of a method for testing an operation of a radiotherapy apparatus. That is, for any radiotherapy apparatus described herein, a method for testing an operation of a radiotherapy apparatus may comprise controlling the radiation source to irradiate a phantom with a beam of radiation, and controlling the first detector to detect the position of the phantom. The beam of radiation comprises a central axis, and the first detector is arranged to detect a position of the central axis. The irradiation of the phantom using the beam of radiation allows for the position of the phantom to be detected using the first detector. The first detector, which comprises a two-dimensional array of pixels, may then generate a two dimensional map of radiation intensity, which may then be used to determine the position of the phantom. The phantom may be, for example, a ball-bearing phantom or the like.

This method may further comprise detecting a position of a leaf of the plurality of leaves of the MLC, using the second detector of the apparatus. The second detector of the apparatus may comprise either an inline detector, or a crossline detector, as described previously. Optionally, the position of the leaf may be detected by controlling the MLC to move the leaf in the beam of radiation while detecting a projection of the leaf using the second detector. In implementations where there is more than one second detector, more than one second detector may be used to detect the projection of the leaf.

The irradiating of the phantom allows for the second detector to generate a one-dimensional map of radiation intensity data, from which the position of a leaf can be determined. Such a one-dimensional map of radiation intensity data may comprise a beam profile of the radiation map in a particular direction. For a leaf extending in the x-direction, the crossline detector generates the corresponding one-dimensional map of radiation intensity which can be used to determine the position of the leaf. For a leaf extending in the y-direction, one or more of the inline detectors may generate the corresponding map of radiation intensity data, from which the position of that lead can be determined.

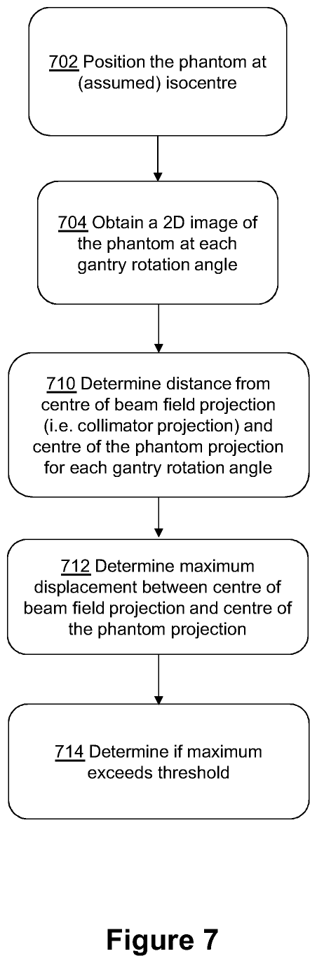

FIG. 7 is a flow chart setting out a method for determining a maximum displacement between the beam axis and centre of the phantom projection for the radiotherapy apparatus 100. The method may be used for isocentre QA.

In the method of FIG. 7, a set of 2D images of a phantom positioned at the radiotherapy apparatus isocentre is obtained. The phantom is generally spherical and may be e.g. a ball-bearing phantom. The method comprises controlling the radiation source to irradiate the phantom with a beam of radiation, and controlling the first detector to detect the position of the phantom. Detecting the position of the phantom may involve obtaining, using the detection device, a set of 2D images of the phantom positioned at the radiotherapy apparatus isocentre for each gantry angle of the set of gantry angles. The location of the isocentre may be slightly different at each gantry rotation angle. For example, an image may be taken at each of the following gantry rotation angles: 0°, 60°, 120°, 180°, 240°, 300°. In this particular example, the set of 2D images would comprise 6 images. Use of the term 2D image is intended to describe an image, a projection, as well as projection or radiation intensity data, as would be understood by the person skilled in the art.

At 702, the phantom is positioned at the isocentre (e.g. the assumed isocentre) for one of the gantry rotation angles of the set of gantry rotation angles. The initial positioning of the phantom may be assisted by lasers suitably positioned to direct visible light along the same path as the treatment beam, and/or by markers on the couch 110.

Once the phantom has been positioned at the treatment beam isocentre for a particular gantry rotation angle, a 2D image is obtained at block 704 using the primary detector. This is repeated for a different gantry rotation angle, until a 2D image of the phantom at the isocentre is obtained for each of the gantry rotation angles of the set of gantry rotation angles.

At block 710, a distance from the centre of beam field projection (i.e. collimator projection), corresponding to the beam axis, and centre of the phantom projection is determined, for each gantry rotation angle. This determination may be carried out by the controller 140 based on the images obtained.

At block 712, a maximum displacement between centre of beam field projection and centre of the phantom projection is determined by comparing the displacement for each gantry angle.

At block 714, it is determined whether the maximum displacement exceeds a threshold displacement. If the threshold is not exceeded, then the system has passed this QA test.

Any implementations, examples, embodiments and the like described herein can be combined.

It will be understood that the description of specific implementations, examples, embodiments and the like is by way of example only and is not intended to limit the scope of the present disclosure. Many modifications of the described embodiments, some of which are now described, are envisaged and intended to be within the scope of the present disclosure.

In some implementations, the radiotherapy apparatus 100 is a combination magnetic resonance imaging (MRI) and linear accelerator.

The radiation head 104 may comprise a heavy metal target towards which high energy electrons are directed. When the electrons strike the target, X-rays are produced in a variety of directions. A primary collimator (not shown) may block X-rays travelling in certain directions and pass only forward travelling X-rays to produce a treatment beam. The X-rays may be filtered and may pass through one or more ion chambers for dose measuring. In some implementations, the source of radiation 107 is configured to emit either an X-ray beam or a particle beam. Such implementations allow the device to provide particle beam therapy, i.e. a type of external beam therapy where particles (e.g. protons or light ions), rather than X-rays, are directed toward the target region.

In some examples, the radiation source and the beam shaping apparatus are provided as a single unit, or alternatively the radiation source and the beam shaping apparatus may be provided separately.

It will be appreciated that the gantry may be replaced with one or more apparatus which allows the radiation head and the beam receiving apparatus to rotate around an axis of rotation.

The detection device may also be described as a portal detection device, radiation detecting means or a detector system.

The detection device may be formed as a detection panel.

The detection device may not be unitary—for example it may include distributed components.

In some embodiments, the detection device is not used for patient imaging, and may not be used at all when a patient is undergoing radiotherapy.

The radiotherapy apparatus can include an image guided radiotherapy system, which provides patient imaging without using the MV beam.

The detection device can be used in, for example, measuring of machine radiation output, measuring of beam shape, measuring of collimator position, measuring of the apparatus isocentre, MLC calibration, apparatus setup and portal dosimetry (the measurement of the exit dose from the patients and subsequent reconstruction of delivered dose on the patient geometry).

The detection device is preferably provided as an integrated portal detector, for example in place of an EPID. Many radiotherapy devices are now equipped with improved patient imaging systems such as kV CBCT or MRI and this makes it unnecessary to use the EPID for patient imaging.

The radiation beam axis may be defined by the geometry of the radiotherapy apparatus e.g. the edge of the central MLC leaves, or the geometric centre of the beam. The radiation beam axis may alternatively be defined as, for example, a point of maximum intensity. The beam profile may be flat (e.g. flat within tolerances).

The radiotherapy apparatus also comprises several other components and systems as will be understood by the skilled person. For example, in order to ensure the radiotherapy apparatus does not leak radiation, appropriate shielding is also provided.

The beam shaping apparatus may comprise an interface ring or other means which is configured to allow the beam shaping apparatus to be attached to the radiation source or other components, for example an ion chamber and/or dosimeter.

In some implementations, the MLC also includes leaf bank actuation means. The leaf bank actuation means is configured to move the entire bank of leaves such that the bank of leaves may be extended into, and withdrawn from, the radiation field. Once the banks are in the correct position, each leaf is individually actuated so as to form the desired shape.

There may be provided any actuator or combination or actuators for controlling the leaves of the MLC and/or the diaphragm blocks.

It is noted that, while it is described herein that the diaphragm blocks define the aperture in the x direction, the diaphragm blocks may instead define the aperture in the y direction (additionally or alternatively to the MLC). The diaphragm may include two pairs of blocks in order to define the aperture in the x and y directions.

The width of the leaves is defined by the design of the MLC and may be between 2 mm and 10 mm when projected onto the isocentric plane.

The MLC in the radiotherapy apparatus may have the capability to rotate around the beam axis. In such cases, the detection device can be configure to rotate in the same way, such that the MLC remains aligned with the detection device.

In other implementations, the MLC is fixed such that it does not rotate around the beam axis, and the detection device is also fixed in alignment with the MLC.

It will be appreciated that all implementations of the detection device include detecting regions and non-detecting regions. The primary and secondary detectors provide detecting regions, with the space in between being non-detecting. Even within detectors there can be non-detecting regions—in particular, in between sensors of the secondary detectors, there may be non-detecting regions. In other examples, the sensors of the secondary detectors may be contiguous (except, for example, where the primary detector is disposed partially within a second detector).

Secondary detectors may be spaced apart from one another, e.g. separated by a non-detecting region.

Any of the secondary detectors and/or the primary detector may be configured to detect a position of each of the movable blocks of the diaphragm.

Although the MLC leaves and detection device are described and illustrated as corresponding to particular x and y directions, it will be appreciated that different orientations can be used (e.g. the MLC leaves can move in the x direction, the y direction, or another direction).

In some embodiments, the peripheral inline detectors are disposed at or near a point of maximum retraction of the MLC leaves.

As described above, preferably the aperture of the primary detector projected to isocentre is also smaller than typical aperture sizes (projected to isocentre) of the beam shaping device used for radiotherapy. However, in some embodiments the aperture of the primary detector projected to isocentre is configured to be the same or bigger than typical aperture sizes (projected to isocentre) of the beam shaping device used for radiotherapy, in order that the primary detector can be used for portal dosimetry.

In some embodiments, the primary detector (and/or the secondary detectors) may be movable relative to the radiation source. The controller may be configured to control movement of the primary detector relative to the radiation source in order to ensure that the beam central axis is incident on the primary detector, and/or a phantom placed at the isocentre can be detected (e.g. imaged in full) by the primary detector. The controller may alternatively or additionally be configured to control movement of the primary detector relative to the radiation source in order to ensure that all of the radiation beam is incident on the primary detector.

For example, the primary detector (and/or the secondary detectors) may be connected to the gantry via servo-controlled linkages that allow x-y movement of the primary detector relative to the gantry. The two translation axes of the linkages may be arranged transverse to the beam direction, so the effect of translating the primary detector is to scan it across the field of the beam. The gantry and/or the linkages may be also able to move the primary detector in a z-direction, i.e. towards or away from the source. The controller can control the position of the primary detector by controlling the servo-controlled linkages.

In an alternative implementation, the phantom may be placed at or substantially at the geometric centre of the treatment volume, and a full 360-degree scan (or single scan) is performed. The primary detector is used to image the (ball-bearing) phantom. The radiation source may be controlled to irradiate the phantom with the beam of radiation, and the primary detector may then be used to detect the position of the phantom. If it is determined, from the images taken, that the ball-bearing phantom is not positioned at the treatment isocentre, then the ball-bearing is moved, and the full 360-degree scan is performed again. This process is repeated until it is determined, from the images taken, that the ball-bearing phantom is located at the treatment isocentre, or the ‘averaged’ treatment isocentre. After this process has been completed, the phantom is imaged at each of the gantry rotation angles of the set of gantry rotation angle in order to obtain the 2D images.

The radiotherapy apparatus may be configured to perform any of the method steps disclosed and may comprise computer executable instructions which, when executed by a processor, cause a processor to perform any of the method steps presently disclosed. Any of the steps that the radiotherapy apparatus is configured to perform may be considered as method steps of the present disclosure and may be embodied in computer executable instructions for execution by a processor.

The controller can be provided by multiple separate controllers (separate in hardware and/or software), and may include distributed components.

The controller is a computing device, computer, processor, or other processing apparatus. The controller may be formed by several discrete processors; for example, the controller may comprise a primary detector processor (also referred to as a primary detector controller), which controls the primary detector; a secondary detector processor (also referred to as a secondary detector controller), which controls the operation of the secondary detector; and a patient support surface processor which controls the operation and actuation of the patient support surface. The controller is communicatively coupled to a memory, e.g. a computer readable medium.

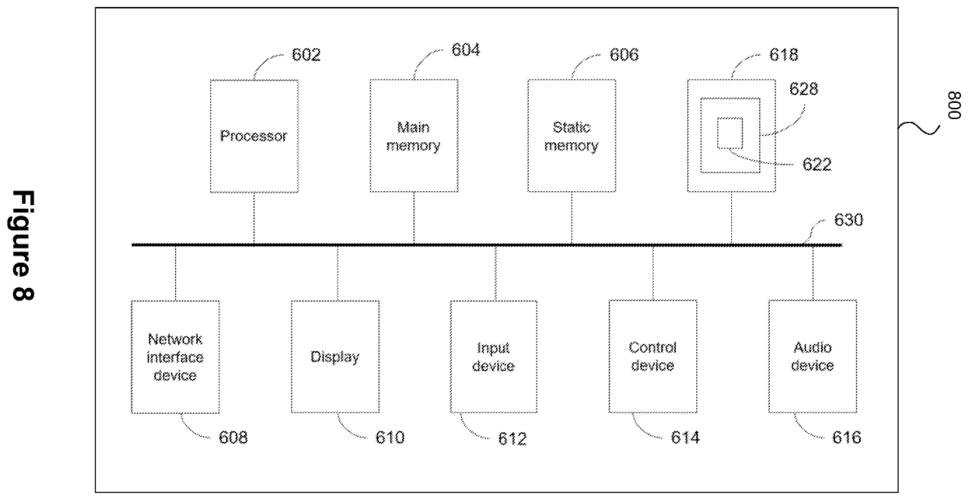

FIG. 8 is a block diagram of one implementation of a computing device 800 within which a set of instructions, for causing the computing device to perform any one or more of the methodologies discussed herein, may be executed. In alternative implementations, the computing device may be connected (e.g., networked) to other machines in a Local Area Network (LAN), an intranet, an extranet, or the Internet. The computing device may operate in the capacity of a server or a client machine in a client-server network environment, or as a peer machine in a peer-to-peer (or distributed) network environment. The computing device may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single computing device is illustrated, the term “computing device” shall also be taken to include any collection of machines (e.g., computers) that individually or jointly execute a set (or multiple sets) of instructions to perform any one or more of the methodologies discussed herein.

The example computing device 800 includes a processing device 602, a main memory 604 (e.g., read-only memory (ROM), flash memory, dynamic random access memory (DRAM) such as synchronous DRAM (SDRAM) or Rambus DRAM (RDRAM), etc.), a static memory 606 (e.g., flash memory, static random access memory (SRAM), etc.), and a secondary memory (e.g., a data storage device 618), which communicate with each other via a bus 630.

Processing device 602 represents one or more general-purpose processors such as a microprocessor, central processing unit, or the like. More particularly, the processing device 602 may be a complex instruction set computing (CISC) microprocessor, reduced instruction set computing (RISC) microprocessor, very long instruction word (VLIW) microprocessor, processor implementing other instruction sets, or processors implementing a combination of instruction sets.

Processing device 602 may also be one or more special-purpose processing devices such as an application specific integrated circuit (ASIC), a field programmable gate array (FPGA), a digital signal processor (DSP), network processor, or the like. Processing device 602 is configured to execute the processing logic (instructions 622) for performing the operations and steps discussed herein.

The computing device 800 may further include a network interface device 608. The computing device 800 also may include a video display unit 610 (e.g., a liquid crystal display (LCD) or a cathode ray tube (CRT)), an alphanumeric input device 612 (e.g., a keyboard or touchscreen), a cursor control device 614 (e.g., a mouse or touchscreen), and an audio device 616 (e.g., a speaker).

The data storage device 618 may include one or more machine-readable storage media (or more specifically one or more non-transitory computer-readable storage media) 628 on which is stored one or more sets of instructions 622 embodying any one or more of the methodologies or functions described herein. The instructions 622 may also reside, completely or at least partially, within the main memory 604 and/or within the processing device 602 during execution thereof by the computer system 800, the main memory 604 and the processing device 602 also constituting computer-readable storage media.

The various methods described above may be implemented by a computer program. The computer program may include computer code arranged to instruct a computer to perform the functions of one or more of the various methods described above. The computer program and/or the code for performing such methods may be provided to an apparatus, such as a computer, on one or more computer readable media or, more generally, a computer program product. The computer readable media may be transitory or non-transitory. The one or more computer readable media could be, for example, an electronic, magnetic, optical, electromagnetic, infrared, or semiconductor system, or a propagation medium for data transmission, for example for downloading the code over the Internet. Alternatively, the one or more computer readable media could take the form of one or more physical computer readable media such as semiconductor or solid state memory, magnetic tape, a removable computer diskette, a random access memory (RAM), a read-only memory (ROM), a rigid magnetic disc, and an optical disk, such as a CD-ROM, CD-R/W or DVD.

In an implementation, the modules, components and other features described herein can be implemented as discrete components or integrated in the functionality of hardware components such as ASICS, FPGAs, DSPs or similar devices.

A “hardware component” is a tangible (e.g., non-transitory) physical component (e.g., a set of one or more processors) capable of performing certain operations and may be configured or arranged in a certain physical manner. A hardware component may include dedicated circuitry or logic that is permanently configured to perform certain operations. A hardware component may be or include a special-purpose processor, such as a field programmable gate array (FPGA) or an ASIC. A hardware component may also include programmable logic or circuitry that is temporarily configured by software to perform certain operations.

Accordingly, the phrase “hardware component” should be understood to encompass a tangible entity that may be physically constructed, permanently configured (e.g., hardwired), or temporarily configured (e.g., programmed) to operate in a certain manner or to perform certain operations described herein.

In addition, the modules and components can be implemented as firmware or functional circuitry within hardware devices. Further, the modules and components can be implemented in any combination of hardware devices and software components, or only in software (e.g., code stored or otherwise embodied in a machine-readable medium or in a transmission medium).

Unless specifically stated otherwise, as apparent from the following discussion, it is appreciated that throughout the description, discussions utilizing terms such as “receiving”, “determining”, “comparing”, “enabling”, “maintaining,” “identifying,” “applying,” “transmitting,” “generating,” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The approaches described herein may be embodied on a computer-readable medium, which may be a non-transitory computer-readable medium. The computer-readable medium may carry computer-readable instructions arranged for execution upon a processor so as to cause the processor to carry out any or all of the methods described herein.

The term “computer-readable medium” as used herein refers to any medium that stores data and/or instructions for causing a processor to operate in a specific manner. Such storage medium may comprise non-volatile media and/or volatile media. Non-volatile media may include, for example, optical or magnetic disks. Volatile media may include dynamic memory. Exemplary forms of storage medium include, a floppy disk, a flexible disk, a hard disk, a solid state drive, a magnetic tape, or any other magnetic data storage medium, a CD-ROM, any other optical data storage medium, any physical medium with one or more patterns of holes, a RAM, a PROM, an EPROM, a FLASH-EPROM, NVRAM, and any other memory chip or cartridge.

It is to be understood that the above description is intended to be illustrative, and not restrictive. Many other implementations will be apparent to those of skill in the art upon reading and understanding the above description. Although the present disclosure has been described with reference to specific example implementations, it will be recognized that the disclosure is not limited to the implementations described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

1. A radiotherapy apparatus comprising:
a radiation source configured to emit a beam of radiation having a central axis;
a multi-leaf collimator, MLC, for shaping the beam of radiation emitted by the radiation source, wherein the MLC comprises a plurality of leaves; and
a detection device for detecting radiation emitted by the radiation source, wherein the detection device comprises:
a first detector arranged to detect a position of the central axis, wherein the first detector comprises a two dimensional array of pixels for generating a two dimensional map of radiation intensity; and
at least one second detector arranged to detect a position of each leaf of the plurality of leaves.

2. The radiotherapy apparatus of claim 1, wherein the first detector has a higher resolution than the at least one second detector.

3. The radiotherapy apparatus of claim 1, wherein the radiotherapy apparatus comprises a volume between the radiation source and the detection device in which a phantom may be placed to be irradiated.

4. The radiotherapy apparatus of claim 3, wherein the first detector is configured to detect the position of the phantom when the phantom is positioned at or near an isocentre of the radiotherapy apparatus.

5. The radiotherapy apparatus of claim 4, wherein the first detector is configured to detect the position of the phantom relative to the isocentre by imaging a projection of the phantom.

6. The radiotherapy apparatus of claim 1, further comprising:
a controller configured to control the radiation source, the MLC, and the detection device, in order to determine a position of the central axis of the beam of radiation with respect to the radiotherapy apparatus and to determine a position of each leaf of the MLC with respect to the radiotherapy apparatus.

7. The radiotherapy apparatus of claim 6, wherein the controller is further configured to control the at least one second detector to a detect a profile of the beam of radiation.

8. The radiotherapy apparatus of claim 1, wherein the at least one second detector is spaced apart from the first detector.

9. The radiotherapy apparatus of claim 8, wherein the first detector and the at least one second detector are separated by at least one non-detecting region of the detection device.

10. The radiotherapy apparatus of claim 1, wherein the first detector has a smaller pitch between pixels than the at least one second detector, thereby having a higher resolution.

11. The radiotherapy apparatus of claim 1, wherein the first detector or each second detector comprises a one dimensional array of sensors.

12. The radiotherapy apparatus of claim 11, wherein each of a plurality of the sensors are aligned with a respective leaf of the MLC in order to detect a position of the respective leaf.

13. The radiotherapy apparatus of claim 11, wherein the detection device comprises at least two second detectors, thereby allowing the detection device to detect each leaf in at least two discrete positions.

14. The radiotherapy apparatus of claim 11, wherein a pitch between sensors is the same as a pitch of the leaves of the MLC when projected to the detection device.

15. The radiotherapy apparatus of claim 11, wherein the at least one second detector comprises two orthogonal second detectors configured to detect a profile of the beam of radiation in two dimensions.

16. The radiotherapy apparatus of claim 1, wherein the MLC and the detection device are disposed in a fixed position relative to one another.

17. The radiotherapy apparatus of claim 16, wherein the MLC and the detection device are provided at opposing sides of a rotatable gantry.

18. The radiotherapy apparatus of claim 17, wherein the first detector is configured to determine a location of the central axis of a field of radiation at each of a plurality of gantry rotation angles, in order to allow determination of an isocentre position for the radiotherapy apparatus.

19. The radiotherapy apparatus of claim 1, wherein the detection device is configured to provide dosimetry data for a dose of radiation delivered to a patient.

20. A method of testing an operation of a radiotherapy apparatus, the radiotherapy apparatus comprising:
a radiation source configured to emit a beam of radiation having a central axis;
a multi-leaf collimator (MLC) for shaping the beam of radiation emitted by the radiation source, wherein the MLC comprises a plurality of leaves; and
a detection device for detecting radiation emitted by the radiation source, wherein the detection device comprises:
a first detector arranged to detect a position of the central axis, wherein the first detector comprises a two dimensional array of pixels for generating a two dimensional map of radiation intensity; and
at least one second detector arranged to detect a position of each leaf of the plurality of leaves;
the method comprising:
controlling the radiation source to irradiate a phantom with the beam of radiation; and
controlling the first detector to detect the position of the phantom.

21. The method of claim 20, further comprising:
detecting a position of a leaf of the plurality of leaves of the MLC using the at least one second detector.

22. The method of claim 21, wherein the position of the leaf is detected by controlling the MLC to move the leaf in the beam of radiation while detecting a projection of the leaf using the at least one second detector.

23. A non-transitory computer-readable medium comprising computer-executable instructions which, when executed by a processor, cause the processor to:
control a radiation source of a radiotherapy apparatus to irradiate a phantom with a beam of radiation; and
control a first detector of the radiotherapy apparatus to detect a position of the phantom, wherein the radiotherapy apparatus comprises:
the radiation source, wherein the radiation source is configured to emit a beam of radiation having a central axis;
a multi-leaf collimator (MLC) for shaping the beam of radiation emitted by the radiation source, wherein the MLC comprises a plurality of leaves; and
a detection device for detecting radiation emitted by the radiation source, wherein the detection device comprises:
the first detector arranged to detect a position of the central axis, wherein the first detector comprises a two dimensional array of pixels for generating a two dimensional map of radiation intensity; and
at least one second detector arranged to detect a position of each leaf of the plurality of leaves.
